(19) United States
(12) Patent Application Publication
(10) Pub. No.: US 2021/0000986 A1
(43) Pub. Date: Jan. 7, 2021

(54) INCLUSION COMPLEXES SUITABLE FOR USE AS A HISTOTRIPSY AGENT

(71) Applicant: T.C ISTANBUL MEDIPOL ÜNIVERSITESI, Istanbul (TR)

(72) Inventors: Yasemin YUKSEL DURMAZ, Istanbul (TR); Tanzeel Ur REHMAN, Istanbul (TR)

(21) Appl. No.: 16/980,540

(22) PCT Filed: Mar. 18, 2019
(86) PCT No.: PCT/TR2019/050171

§ 371 Date: Sep. 14, 2020

(30) Foreign Application Priority Data
Mar. 16, 2018 (TR) 2018/03803

(51) Int. Cl. A61K 49/22; A61K 47/69

(52) U.S. Cl. CPC A61K 49/228; A61K 9/0019; A61K 47/6951

(57) ABSTRACT

Disclosed are inclusion complexes suitable for use as histotripsy agents, the methods used in the preparation of the inclusion complexes, and the use of the complexes as histotripsy agents or for drug delivery.

### TECHNICAL FIELD

The present invention relates to inclusion complexes for use as histotripsy agents, the methods used in the preparation of said complexes, and the use of the complexes according to the invention as histotripsy agents or for drug delivery.

### STATE OF THE ART

Histotripsy is the mechanical disruption of the cell by acoustic cavitation mechanism using high frequency ultrasound (US) signals within microseconds. These ultrasound signals form a bubble cloud from the bubbles of gas already present in the body in dissolved form. As a result of the fragmentation of this cloud through collecting sufficient energy (cavitation), mechanical disintegration/damage occurs in the tissue they are in. Very high pressure is required for cavitation.

Histotripsy is a new method that is intended to be used for the destruction of tumor tissues in cancer cases because of the damage it imparts to the tissue. However, in the absence of a histotripsy agent, a pressure of about 28 MPa to 30 M Pa is required to obtain a gas cloud from the gas bubbles, a pressure of this magnitude is capable of causing damage even in tumor-free healthy tissue.

Based on this aim, nanodroplet-mediated histotripsy has recently been developed. In said process, a perfluorocarbon (PFK), for example perfluoropentane encapsulated polymeric nanodroplets, are used. When this agent enters the tumor, instead of the gas bubbles in the tissue, the perfluoropentane within these nanodroplets serves as the core of gas cloud (cavitation) formation. With this method, it is seen that the pressure required to form the cavitation has decreased from 28 MPa to 7 MPa, thus preventing damage to the healthy tissue during the application.

The present method involves complex steps for the preparation of a polymer consisting of three blocks as the main component of nanodroplets, which requires improved synthesis ability and expertise. Another point is that it is not possible to determine the amount of PFK which is encapsulated to the nuclei of the nanodroplets. By a secondary characterization method, the concentration of nanodroplets is determined as the number of nanodroplets per mL and the applied dose can be calculated from this value, but this does not mean that the amount of PFK is determined because the amount of PFK differs not only by the number but also by the size and size distribution of the nanodroplets. Furthermore, the nanodoplets are the only known agents which can be used as histotripsy agents, and it is necessary to develop new agents which are easy to prepare and user-friendly can be used as an alternative to these agents.

With the present invention, the inventors aim to develop novel histotripsy agents which are easy to prepare.

The inventors also intend to develop novel histotripsy agents that provide ease of use and storage.

The inventors also aim to develop histotripsy agents in which concentration can be readily determined.

### DETAILED DESCRIPTION OF THE INVENTION

The present invention is related to host-guest inclusion complexes comprising a host molecule comprising alpha-cyclodextrin, beta-cyclodextrin, gamma-cyclodextrin, or water-soluble derivatives thereof or cucurbituryl, pillarene, calixarene that are made biocompatible and a guest molecule selected from C3-C8 perfluorocarbon derivatives.

The term “alpha-cyclodextrin” as used herein refers to a polysaccharide consisting of six (6) glucose units linked to each other by alpha 1-4 bonds. The molecule has a conical structure and it is hydrophobic in the inside and hydrophilic in the outside.

The term “beta-cyclodextrin” as used herein refers to a polysaccharide consisting of seven (7) glucose units covalently bound to each other. The molecule has a conical structure and it is hydrophobic in the inside and hydrophilic in the outside.

The term “gamma-cyclodextrin” as used herein refers to a polysaccharide consisting of eight (8) glucose units covalently linked to each other. The molecule has a conical structure and it is hydrophobic in the inside and hydrophilic in the outside.

As used herein, the term “made biocompatible” or “biocompatible” refers to the fact that the molecules in question (cucurbituryl, pillarene, calixarene) have been modified in such a way that they do not cause any immune reaction in the body, and do not cause any cytotoxic or hemolytic effects. Said modification may be made with any protein or amino acid or a polymer, for example poly (ethylene glycol) (PEG) chains of various sizes. In this context, the term “cucurbituryl, pillarene and calixarene that are made biocompatible” also includes PEGylated cucurbituryl, PEGylated pillarene or PEGylated calixarene.

As used herein, the term “cucurbituryl” refers to macrocyclic molecules composed of glycoluryl monomers linked together by methylene bridges. These molecules can be of different sizes depending on the number of glycoluryl units comprised in them. The cucurbituryl molecules used within the scope of the invention can comprise 5 or 6 or 7 or 8 glycoluryl units.

The term “calixarene” as used herein refers to organic macrocyclic molecules composed of phenol groups linked together by short hydrocarbon bridges.

The term “pillarene” as used herein refers to macrocyclic molecules composed of hydroquinone units interconnected by methylene bridges in the para position. They may be of different sizes according to the number of hydroquinone units in said molecules. The pillarene molecules used in the scope of the invention may comprise 5 or 6 or 7 or 8 or 9 or 10 hydroquinone units. The terms “pillarene” and “pillararene” as used in the context of the invention are the same and can be used interchangeably to express the structure described above.

The term “host molecule” as used in the context of the invention refers to the alpha-cyclodextrin or beta-cyclodextrin or gamma-cyclodextrin or biocompatible cucurbituryl or biocompatible calixarene or modified alpha-cyclodextrin or modified beta-cyclodextrin or modified gamma-cyclodextrin molecule and may be used interchangeably within the scope of the present invention.

The term “C3-C8 perfluorocarbon derivative” used in the context of the invention comprises octafluoropropane, decafluorobutane, perfluoropentane, perfluorohexane, perfluoroheptane and perfluorooctane molecules and branched saturated fluorocarbon structures bearing C3-C8 carbon. In a preferred embodiment of the invention, perfluorohexane is used as the C3-C8 perfluorocarbon derivative.

In another aspect, the present invention is related to a host-guest inclusion complex comprising a host molecule selected from alpha-cyclodextrin, beta-cyclodextrin, gamma-cyclodextrin or biocompatible cucurbituryl, pillarene, or calixarene and a guest molecule selected from C3-C8 perfluorocarbon derivatives suitable for use as a histotripsy agent and/or as drug targeting agent or as ultrasound imaging agent In a preferred embodiment of the invention, the invention relates to beta-cyclodextrin and perfluorohexane host-guest inclusion complexes suitable for use as the histotripsy agent and/or as the drug targeting agent or as ultrasound imaging agent.

The term “guest-host inclusion complex” as used in the context of the invention refers to the encapsulation of a host molecule, e.g., beta-cyclodextrin, by non-covalent interaction of a guest molecule, for example, perfluorohexane. Within the scope of the invention, the terms “guest-host inclusion complex”, “inclusion complex” or “host-guest inclusion complex” are of the same meaning and can be used interchangeably.

Beta-cyclodextrin is a circular ring-shaped molecule with a hydrophilic outer surface and a hydrophobic inner surface consisting of 7 sugar units. The fact that the use of this molecule is safe has been confirmed by the US Food and Drug Administration (FDA).

The hydrophobic inner surface of beta-cyclodextrin interacts with perfluorohexane that is a hydrophobic molecule and encapsulates it, and due to the hydrophilic nature of the outer surface of the beta-cyclodextrin, transmission of the resulting inclusion complex to the target tissue in the body's hydrophilic environment is provided.

The host molecule used in accordance with the invention can be used, for example, without any modification of the beta-cyclodextrin, or by modification with a hydrocarbon, such as methyl, ethyl, propyl, hydroxy propyl, sulfobutyl ether, preferably methyl group, or by modification with a water-soluble polymer, such as poly (ethylene glycol) or tertiary polyamines. or chitosan, dextran, hyaluronic acid, poly (oxazoline), poly (N-(2-hydroxypropyl) methacrylamide (HPMA), preferably poly (ethylene glycol).

In a preferred embodiment of the invention, the methyl group modified host is used to increase the host solubility.

In a particularly preferred embodiment of the invention, methyl group modified/methylated beta-cyclodextrin is used to increase the solubility of beta-cyclodextrin.

As used herein, the term “modified” means that 10 to 100%, preferably 20 to 90%, most preferably 30 to 80% of the modifiable groups on the molecule are modified by said modification groups. In other words; Modifiable groups present on said host molecules may be present such that 10%, 20%, 30%, 40%, 50%, 60%, 70%, 80%, 90% or 100% of the modifiable groups are modified with a modification group.

Thus, the term “beta-cyclodextrin” as used in the invention includes any unmodified beta-cyclodextrin and a hydrocarbon modified beta-cyclodextrin, for example with a methyl group, i.e. methylated beta-cyclodextrin. The hydrocarbon modified host molecule of the invention, for example beta-cyclodextrin, may be purchased from commercial sources, modified with any hydrocarbons, for example methyl group, or may be prepared in laboratory, using methods known to those skilled in the art. In the context of the invention, the terms “methylated” and “methyl group modified” are used interchangeably.

A preferred embodiment of the invention relates to methylated beta-cyclodextrin and perfluorohexane guest-host inclusion complexes.

Perfluorohexane; is a member of the organofluorine family and has stable CF bonds. The substance is not metabolized in the body, but can be simply excreted by inhalation.

Perfluorohexane is in liquid form at room temperature, its boiling point is 56° C. The fact that it has a low boiling point is advantageous for the use of this substance as a histotripsy agent. In this way, perfluorohexane evaporates with low pressure and creates a cloud of gas to provide cavitation in the tissue. Furthermore, perfluorohexane is an ultrasound contrast agent. In this way, whether or not the beta-cyclodextrin-perfluorohexane inclusion complexes reach the tumor tissue can easily be seen by ultrasound and the cavitation process can be initiated after the agents reach the target tissue. Furthermore, beta-cyclodextrin is predicted to penetrate the tumor tissue better than known histotripsy agents and thus provide a more effective cavitation because of its small size and uniform structure.

In another aspect, the invention relates to a host-guest inclusion complex comprising a host molecule selected from alpha-cyclodextrin, beta-cyclodextrin, gamma-cyclodextrin or biocompatible cucurbituryl, pillarene or calixarene modified by a targeting agent, and a guest molecule selected from C3-C8 perfluorocarbon derivatives.

Furthermore, the invention relates to beta-cyclodextrin or methylated beta-cyclodextrin and perfluorohexane guest-host inclusion complexes modified with a targeting agent.

Modification with the targeting agent as mentioned herein is obtainable by conjugation of a host molecule selected from the group comprising alpha-cyclodextrin, beta-cyclodextrin, gamma-cyclodextrin or biocompatible cucurbituryl, pillarene or calixarene to a targeting agent. In a preferred embodiment of the invention, the host molecule is obtained by conjugation of the free —OH group present on the outer surface of the alpha-cyclodextrin or beta-cyclodextrin or gamma-cyclodextrin molecule with the targeting agent.

As used herein, the term “targeting agent” refers to molecules that tend to bind to various specific target tissues within the body. In other words, the targeting agents refer to molecules which have a tendency to bind to cells having specific receptors.

Targeting agents that may be used in the context of the invention may be selected from antibodies, antibody fragments, or various peptides.

One embodiment of the invention relates to a process for preparing inclusion complexes comprising a host molecule selected from alpha-cyclodextrin, beta-cyclodextrin, gamma-cyclodextrin or biocompatible cucurbituryl, pillarene or calixarene, and a guest molecule selected from C3-C8 perfluorocarbon derivatives wherein said method comprises the steps of;
a. Dissolving the host molecule, for example beta-cyclodextrin, in a suitable solvent
b. Cooling of the obtained solution to a certain temperature
c. Addition of a guest molecule selected from C3-C8 perfluorocarbon derivatives, e.g. perfluorohexane, to the cooled solution,
d. Obtaining an inclusion complex such as a beta-cyclodextrin-perfluorohexane by separating the solid and liquid parts of the formed precipitate and drying the solid portion.

As used herein, the term “a solvent” refers to any organic solvent that will dissolve the reagents used during the reaction.

In one embodiment of the invention, the appropriate solvent used in step a) may be an organic solvent or water or any aqueous solution. In a preferred embodiment of the invention water is used as solvent.

In one embodiment of the invention, in step a) the solution of the beta-cyclo-dextrin solution may be heated to a certain temperature to provide dissolution, the solution is preferably heated to 70-90° C., particularly preferably to 80° C.

In one embodiment of the invention, in step b) the solution is cooled to a temperature of 35° C. to 55° C., preferably 45° C.

Another embodiment of the invention relates to a process for preparing methylated beta-cyclodextrin perfluorohexane inclusion complexes according to the invention wherein said method comprises the steps of;
a. Dissolution of beta-cyclodextrin molecule in a suitable organic solvent
b. Addition of an inorganic salt to the resulting solution
c. Addition of dimethyl carbonate to the obtained solution
d. Stirring the resulting mixture at room temperature
e. Filtration of the solution to remove the solids and then evaporation of the volatile components to obtain methylated beta-cyclodextrin.
f. Dissolution of the obtained methylated beta-cyclodextrin in a suitable solvent
g. Stirring the solution after adding perfluorohexane to the resulting solution.
h. Separation of the formed solids from the liquids and drying the solids to obtain inclusion complexes of methylated beta-cyclodextrin-perfluorohexane.

The invention further relates to a use of host-guest inclusion complex comprising a host molecule comprising alpha-cyclodextrin, beta-cyclodextrin, gamma-cyclodextrin or biocompatible cucurbituryl, pillarene or calixarene, and a guest molecule selected from C3-C8 perfluorocarbon derivatives for therapeutic purposes.

In one aspect, the invention relates to beta-cyclodextrin perfluorohexane guest-host inclusion complexes for use in therapeutic purposes.

In an aspect, the invention relates to methylated beta-cyclodextrin perfluorohexane guest-host inclusion complexes for a therapeutic use.

In a preferred embodiment of the invention, the invention relates to the host-guest inclusion complexes comprising a host molecule selected from alpha-cyclodextrin, beta-cyclodextrin, gamma-cyclodextrin, or biocompatible cucurbituryl, pillarene or calixarene and a guest molecule selected from C3-C8 perfluorocarbon derivatives for use in the treatment of cancer

In another embodiment of the invention, beta-cyclodextrin perfluorohexane host-guest inclusion complexes are for use in the treatment of cancer.

In another embodiment of the invention, methylated beta-cyclodextrin perfluorohexane guest-host inclusion complexes are for use in the treatment of cancer.

The invention further relates to host-guest inclusion complexes comprising a host molecule selected from alpha-cyclodextrin, beta-cyclodextrin, gamma-cyclodextrin or biocompatible cucurbituryl, pillarene or calixarene, and a guest molecule selected from C3-C8 perfluorocarbon derivatives for use as histotripsy agent.

The invention also relates to beta-cyclodextrin perfluorohexane guest-host inclusion complexes for use as a histotripsy agent.

The invention also relates to the use of methylated beta-cyclodextrin perfluorohexane host-guest inclusion complexes for use as a histotripsy agent.

The invention further relates to host-guest inclusion complexes comprising a host molecule selected from alpha-cyclodextrin, beta-cyclodextrin, gamma-cyclodextrin or biocompatible cucurbituryl, pillarene or calixarene, and a guest molecule selected from C3-C8 perfluorocarbon derivatives for use in diagnostic purposes.

The invention also relates to the beta-cyclodextrin perfluorohexane guest-host inclusion complexes for use in diagnostic purposes.

The invention further relates to methylated beta-cyclodextrin perfluorohexane guest-host inclusion complexes for use in diagnostic purposes.

In a preferred embodiment of the invention, the invention relates to the host-guest inclusion complexes comprising a host molecule selected from alpha-cyclodextrin, beta-cyclodextrin, gamma-cyclodextrin, or biocompatible cucurbituryl, pillarene or calixarene and a guest molecule selected from C3-C8 perfluorocarbon derivatives for use in the diagnosis of various types of cancer

The invention also relates to the beta-cyclodextrin perfluorohexane guest-host inclusion complexes for use in the diagnosis of various types of cancer.

The invention also relates to the methylated beta-cyclodextrin perfluorohexane guest-host inclusion complexes for use in the diagnosis of various types of cancer.

The invention further relates to host-guest inclusion complexes comprising a host molecule selected from alpha-cyclodextrin, beta-cyclodextrin, gamma-cyclodextrin or biocompatible cucurbituryl, pillarene or calixarene, and a guest molecule selected from C3-C8 perfluorocarbon derivatives for use in imaging purposes.

The invention also relates to the beta-cyclodextrin perfluorohexane host-guest inclusion complexes for use in imaging purposes.

The invention further relates to methylated beta-cyclodextrin perfluorohexane guest-host inclusion complexes for use in imaging purposes.

In a preferred embodiment of the invention, the invention relates to the host-guest inclusion complexes comprising a host molecule selected from alpha-cyclodextrin, beta-cyclodextrin, gamma-cyclodextrin, or biocompatible cucurbituryl, pillarene or calixarene and a guest molecule selected from C3-C8 perfluorocarbon derivatives for use in ultrasound imaging.

In a particularly preferred embodiment of the invention, the invention relates to beta-cyclodextrin-perflorohexane inclusion complexes for use in ultrasound imaging.

In a particularly preferred embodiment of the invention, the invention relates to methylated beta-cyclodextrin perfluorohexane inclusion complexes for use in ultrasound imaging.

In a preferred embodiment of the invention, the invention relates to the host-guest inclusion complexes comprising a host molecule selected from alpha-cyclodextrin, beta-cyclodextrin, gamma-cyclodextrin, or biocompatible cucurbituryl, pillarene or calixarene and a guest molecule selected from C3-C8 perfluorocarbon derivatives for use in the imaging of various cancer types.

In a particularly preferred embodiment of the invention, the invention relates to the beta-cyclodextrin perfluorohexane inclusion complexes for use in the imaging of various types of cancer.

In a particularly preferred embodiment of the invention, the invention relates to the methylated beta-cyclodextrin perfluorohexane inclusion complexes for use in the imaging of various types of cancer.

The term “cancer” as used herein refers to malignant tumors or a physiological condition characterized by uncontrolled cell growth. Cancer examples include, but are not limited to, carcinoma, lymphoma, blastoma sarcoma, and leukemia.

Carcinoma, as used herein, refers to a cancer type of epithelial cells.

Lymphoma, as used herein, describes a cancer type that develops from lymphocytes.

Blastoma, as used herein, refers to a cancer type developed from precursor cells, also known as blast cell.

Sarcoma, as used herein, refers to a cancer type arising from altered cells of mesenchymal origin.

Leukemia, as used herein, refers to a cancer type originating in the bone marrow and causing a high number of abnormal white blood cell formation.

More specific examples of cancer types include breast cancer, prostate cancer, colorectal cancer, skin cancer, small cell lung cancer, non-small cell lung cancer, mesothelioma, gastrointestinal cancer, pancreatic cancer, glioblastoma, vulva cancer, cervical cancer, endometrial carcinoma, ovarian cancer, liver cancer, hepatoma, bladder cancer, kidney cancer, salivary gland carcinoma, thyroid cancer and various head and neck cancers.

The invention further relates to pharmaceutical compositions comprising a host-guest inclusion complex comprising a host molecule comprising alpha-cyclodextrin, beta-cyclodextrin, gamma-cyclodextrin or biocompatible cucurbituryl, pillarene or calixarene, and a guest molecule selected from C3-C8 perfluorocarbon derivatives.

The invention also discloses pharmaceutical compositions comprising beta-cyclodextrin perfluorohexane guest-host inclusion complexes.

In a preferred embodiment of the invention, the pharmaceutical compositions comprising the host-guest inclusion complex comprising the host molecule selected from the group consisting of alpha-cyclodextrin, beta-cyclodextrin, gamma-cyclodextrin or biocompatible cucurbituryl, pillarene or calixarene and a guest molecule selected from C3-C8 perfluorocarbon derivatives further comprise least one auxiliary agent in addition to the inclusion complex of the invention.

In a preferred embodiment of the invention, pharmaceutical compositions comprising beta-cyclodextrin perfluorohexane host-guest inclusion complexes comprise at least one auxiliary agent in addition to the inclusion complex according to the invention.

Said auxiliary agent can be used for the enabling the pharmaceutical composition to meet criteria such as solubility, distribution, dosage uniformity, etc. and it does not have any pharmaceutical activity.

Pharmaceutical compositions containing inclusion complexes according to the invention can be present in any dosage form which exists in the state of the art. In a preferred embodiment of the invention, the dosage forms according to the invention are in injectable form. The injectable forms may be prepared in particular for intravenous, intraperitoneal, intratracheal administration.

The invention will now be described by way of example only with reference to the following examples, which are intended to be exemplary only and are not to be construed in any way as limiting the scope of the invention.

### EXAMPLES

### Example 1: Preparation of Beta-Cyclodextrin Perfluorohexane Inclusion Complex

50 mg of beta-cyclodextrin is mixed with 1 mL of distilled water and heated to 80° C. and stirred. After complete dissolution of the beta-cyclodextrin, the solution is cooled to 45° C. Next, perfluorohexane is added in molar ratios of 1, 2, 20 or 50 fold. The mixture is stirred at 45° C. overnight. The mixture is then cooled to 4° C. and centrifuged at 5000 rpm for one hour. The liquid portion is discarded and the resulting solids are dried under vacuum.

### Example 2: Preparation of Methylated Beta-Cyclodextrin Perfluorohexane Inclusion Complex

Dissolve 3 g of beta-cyclodextrin in 60 ml of DMF in a double neck flask with a condenser. After complete dissolution of the beta-cyclodextrin, 8.6 g of K₂ CO₃ are added to the mixture. 8 mL of anhydrous dimethyl carbonate is then added dropwise to the mixture, and the resulting reaction mixture is stirred at room temperature overnight.

The mixture is then centrifuged for 5 minutes at 2000 rpm to remove the catalyst. The solvent and excess dimethyl carbonate are removed by distillation under reduced pressure. The residue is then recrystallized in acetone and the precipitate formed is washed with diethylether. The obtained solid is filtered and dried under vacuum.

To obtain the methylated beta-cyclodextrin perfluorohexane inclusion complex, firstly 50 mg of methylated beta-cyclodextrin is dissolved in 1 ml of water at room temperature, followed by the addition of perfluorohexane in molar ratios of 5, 10 or 50 fold. The resulting solution is stirred for 24 hours. The reaction mixture is then centrifuged at 5000 rpm for 10 minutes, the liquid portion is discarded, and the solid which precipitates is dried under reduced pressure.

### Example 3: Cell Viability Tests

The cell viability test is important to determine how the methylated beta-cyclodextrin perfluorohexane inclusion complex interacts with cells in the body.

Inclusion complexes according to the invention were tested using kidney HEK-293T cells from the reticulo-endothelial system organs responsible for cleansing blood in the body and removing various particles and antigens from blood flow and forming inflammatory mediators against immunological stimuli.

For this purpose, beta-cyclodextrin (BCD), methylated beta-cyclodextrin (MCD) and methylated beta-cyclodextrin and perfluorohexane inclusion complex (MIC) solutions at a concentration of 0.1 mg/mL, 0.5 mg/mL and 1 mg/mL were prepared. In addition, perfluorohexane (PFH) at amounts of 0.1 μL, 0.13 μL, 0.15 μL were also tested on the same cells. The amount of perfluorohexane tested was based on the amount of perfluorohexane in 1 mg/mL MIC. It was determined that in 1 mg/mL MIC there is 0.15 μL of perfluorohexane. It was found that in lower concentrations of inclusion complexes, for example in 0.1 mg/ml and 0.5 mg/mL concentrations it was found that there is respectively 0.015 μL and 0.075 μL perfluorohexane and because these amounts are very low the experiments were made with amounts of 0.1 μL and 0.13 μL perfluorohexane.

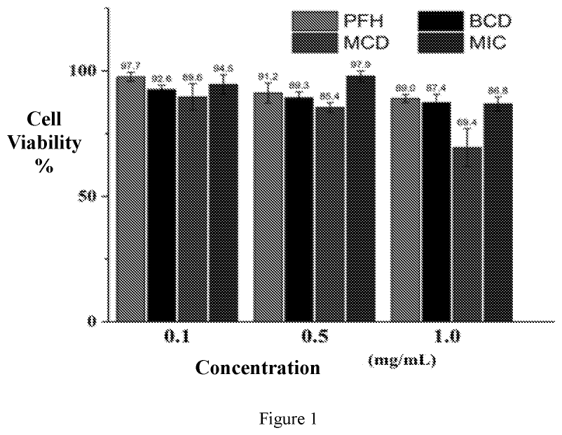

The results are shown in FIG. 1.

As shown in this graph, 0.1 mg/ml MIC inclusion complex showed 94.5% cell viability, when the concentration is increased to 1 mg/mL, the cell viability decreases to 86.8%, indicating that there was no significant decrease in cell viability despite a 10-fold increase in concentration.

On the other hand, perfluorohexane shows 89% cell viability at the maximum concentration of 0.15 μL. This indicates that this agent is not toxic.

### Example 4: Dynamic Light Scattering (DLS) Testing

The size of the complex plays an important role in determining the threshold for histotripsy. Small size particles are more easily injected and they accumulate highly in tumor tissue. Particularly considering that the permeable vascular tissue in the tumor tissue allows passage of the particles within 200 nm and below into the tumor tissue, it is concluded that the particles having a size of less than 200 nm and less are more effective in drug transport or accumulation in the tumor.

In the state of the art, the size of the existing nanodroplets was reported to be 204 nm, which is considered to be at the upper limit and a lower size agent would perform better in the tumor tissue.

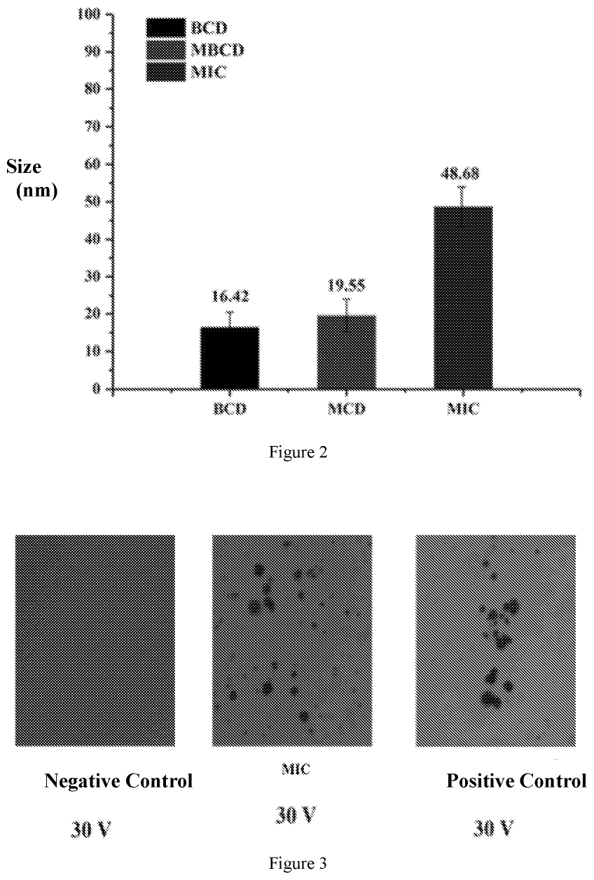

The results obtained in the DLS tests are shown as a graph in FIG. 2.

Herein BCD is beta-cyclodextrin; MBCD is methylated beta-cyclodextrin and MIC refers to the inclusion complex of methylated beta-cyclodextrin and perfluorohexane.

As can be seen from FIG. 2, the beta-cyclodextrin is 16.42 nm in size, methylated beta-cyclodextrin has a size of 19.55 nm and the methylated beta-cyclodextrin and the perfluorohexane inclusion complex is 48.68 nm in size as such the complex has a particle size that is quite below the threshold value of 200 nm that is necessary for entering into the tumor tissue. Because of this feature, it is concluded that the inclusion complexes according to the invention are superior to the histotripsy agents in the form of nanodroplets form the prior art. Another advantage is provided at the point of determining the amount of PFK which provides the main activity of histotripsy. Since the formation of the host-guest complex can be carried out with a high value of 98%-100%, the molar amount of the inclusion complex will be equal to the molar amount of the PFK guest molecule. This situation is important in terms of practicality and reliability.

### Example 5: Measurement of Histotripsy Threshold Value in Agarose Phantom as Tissue Simulator

In the literature, it has been explained that a pressure of 26-30 MPa is required for cavitation in tissues and tissue mimicking environments.

In the first experiment made for this purpose, 30V voltage and 500 kHz transformer were used. Three different environments were prepared to be observed under the same conditions. The first of these is a negative control medium that does not contain any histotripsy agents, another is a positive control medium containing only PFH, and the latter is the medium containing the methylated beta-cyclodextrin perfluorohexane inclusion complex (MIC) according to the invention.

After performing the experiment, high-speed camera images were detected, said images being given in FIG. 3. As can be seen from the images, there is no cavitation in the uncontrolled negative control medium, whereas cavitation occurs in the example where the inclusion complexes according to the invention are used.

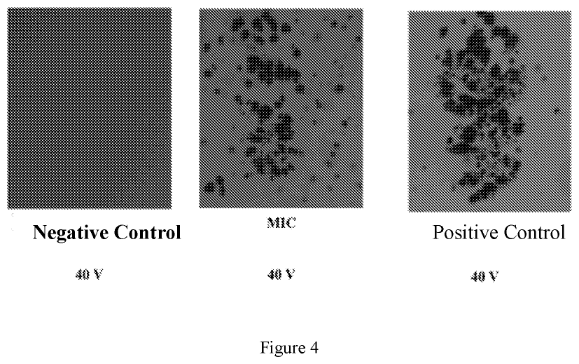

In the second experiment, 40V voltage and 500 kHz transformer were used. For this test, the positive and negative control media whose details are given above and the media containing the MIC according to the invention were used. In this experiment, no cavitation is observed in the negative control medium that does not contain histotripsy agent and cavitation is observed in the medium containing MIC, it is also observed that the formation of cavitation is more than in the 30V environment (FIG. 4).

As a result of these experiments; the fact that there is no cavitation in negative control environments indicates that the pressure generated is less than 26-30 MPa. In the same conditions, the cavitation of the inclusion complexes according to the invention shows that the invention provides cavitation formation at lower pressure values as intended so as not to damage the surrounding healthy tissue.

1. Host-guest inclusion complexes comprising beta-cyclodextrin or methylated beta-cyclodextrin as a host molecule and perfluorohexane or perfluoropentane as a guest molecule.

2. Inclusion complex according to claim 1, characterized in that the host molecule is beta-cyclodextrin.

3. (canceled)

4. The inclusion complex according to claim 1, characterized in that the guest molecule is perfluorohexane.

5. (canceled)

6. (canceled)

7. (canceled)

8. (canceled)

9. An inclusion complex according to claim 1, characterized in that said complex is a methylated beta-cyclodextrin and a perfluorohexane host-guest inclusion complex.

10. An inclusion complex according to claim 1, characterized in that it is modified with a targeting agent.

11. An inclusion complex according to claim 10, wherein the targeting agent is selected from a group comprising antibodies, antibody fragments, or various peptides.

12. A method for preparing an inclusion complex according to claim 1, characterized in that said method comprises the steps of:
a. dissolving the host molecule in a suitable solvent;
b. cooling of the obtained solution to a certain temperature;
c. addition of perfluorohexane or perfluoropentane into the cooled solution;
d. obtaining an host molecule perfluorohexane or perfluoropentane inclusion complex by separating the solid and liquid parts of the formed precipitate and drying the solid portion.

13. A method for preparing an inclusion complex according to claim 12, characterized in that said method comprises the steps of:
a. dissolution of beta-cyclodextrin in a suitable solvent;
b. cooling of the obtained solution to a certain temperature;
c. adding perfluorohexane to the cooled solution;
d. obtaining a betacyclodextrin-perfluorohexane inclusion complex by separating the solid and liquid parts of the formed precipitate and drying the solid portion.

14. A method according to claim 12, wherein in step a) the appropriate solvent is selected from an organic solvent or water or any aqueous solution.

15. The method according to claim 14, wherein water is used as the solvent.

16. A method according to claim 12, characterized in that in step a) the mixture is heated to 70-90° C. to provide dissolution.

17. A method according to claim 12, characterized in that in step b) the solution is heated to a temperature of 35° C. to 55° C.

18. A method for preparing an inclusion complex according to claim 1, characterized in that said method comprises the steps of:
a. dissolving the host molecule in a suitable solvent;
b. addition of an inorganic salt to the resulting solution;
c. addition of dimethyl carbonate to the obtained solution;
d. stirring the resulting mixture at room temperature;
e. filtration of the solution to remove the solids and then evaporation of the volatile components to obtain methylated host molecule;
f. dissolution of the obtained methylated host molecule in a suitable solvent;
g. stirring the solution after adding C3-C8 perfluorocarbon to the resulting solution;
h. Separation of the formed solids from the liquids and drying the solids to obtain inclusion complexes of methylated host molecule-C3-C8 perfluorocarbon.

19. A method for preparing an inclusion complex according to claim 18, characterized in that said method comprises the steps of:
a. dissolution of beta-cyclodextrin in a suitable organic solvent;
b. addition of an inorganic salt to the resulting solution;
c. adding dimethyl carbonate to the obtained solution;
d. stirring the resulting mixture at room temperature;
e. filtration of the solution to remove the solids and then evaporation of the volatile components to obtain methylated beta-cyclodextrin;
f. dissolution of the obtained methylated beta-cyclodextrin in a suitable solvent g. Stirring the solution after adding perfluorohexane to the resulting solution;
h. separation of the formed solids from the liquids and drying the solids to obtain inclusion complexes of methylated betacyclodextrin-perfluorohexane.

20. (canceled)

21. Inclusion complexes according to claim 1, for use in the treatment of cancer.

22. (canceled)

23. Inclusion complexes according to claim 1, for use in diagnosis of various cancer types.

24. (canceled)

25. Inclusion complexes according to claim 1 for use as an ultrasound imaging agent.

26. Inclusion complexes according to claim 1, for use as histotripsy agents.

27. A Pharmaceutical composition comprising inclusion complexes according to claim 1.

28. A pharmaceutical composition according to claim 27, wherein the inclusion complex comprises at least one excipient.

29. A pharmaceutical composition according to claim 27, characterized in that it is prepared in a suitable dosage form.

30. A pharmaceutical composition according to claim 29 wherein it is in an injectable form.

31. A pharmaceutical composition according to claim 30 wherein it is suitable for intravenous, intraperitoneal, intratracheal administration.
